(19) United States
(12) Patent Application Publication
(10) Pub. No.: US 2026/0290511 A1
(43) Pub. Date: Sep. 24, 2026

(54) CONTROLLING HEALTH-RELATED DEVICES USING PHYSIOLOGICAL BIOMARKERS

(71) Applicants: Adam Fischer, San Diego, CA (US); Tyler Fish, San Diego, CA (US); Jeffrey H. Fischer, San Diego, CA (US)

(72) Inventors: Adam Fischer, San Diego, CA (US); Tyler Fish, San Diego, CA (US); Jeffrey H. Fischer, San Diego, CA (US)

(21) Appl. No.: 19/568,993

(22) Filed: Mar. 17, 2026

Related U.S. Application Data
(60) Provisional application No. 63/773,470, filed on Mar. 18, 2025.

(51) Int. Cl. G16B 40/30; G16H 50/20; G16H 50/30

(52) U.S. Cl. CPC G16B 40/30; G16H 50/20; G16H 50/30

(57) ABSTRACT

The present disclosure relates generally to systems, apparatus, and methods, using non-transitory computer-readable storage media for controlling operation of a health-related device based on physiological data obtained from or about a user. More particularly, the disclosure provides a physiological data-driven control method and apparatus in which biomarker measurements are used to preset and adjust one or more operational characteristics of a therapeutic device during use by the user. In this disclosure, the terms biomarker and biometric are distinguished. Biomarkers are biological, medical indicators (e.g., blood pressure, skin temperature, etc.) used to assess physiological state, condition, or health status, whereas biometrics are unique, measurable physical traits (e.g., fingerprints, facial recognition) used for identification and security.

### CROSS-REFERENCE TO RELATED APPLICATIONS

This application claims the benefit of United States Provisional Patent Application No. 63/773,470 filed on Mar. 18, 2025, and United States Provisional Patent Application No. 63/930,554, filed Dec. 3, 2025, which are incorporated by reference herein in their entirety.

### FIELD OF THE INVENTION

The present disclosure relates generally to health-related device control systems and, more particularly, to systems, apparatus, and methods for controlling the operation of a health-related device based on physiological data obtained from a user.

### BACKGROUND OF THE INVENTION

Health-related devices including health maintenance and health improvement devices are increasingly used for therapeutic, rehabilitative, and wellness purposes. Such devices may include, for example, thermal therapy systems, cold plunge devices, saunas (conventional, infrared, hybrid), biochromatic therapy, infrared blankets, massage systems, electrical and electronic stimulus systems, sonic therapy, and in general recovery devices, rehabilitation apparatuses, and other equipment designed to expose a user to controlled physical or environmental conditions intended to produce therapeutic, wellness and physiological improvement benefits. Conventional health-related devices are typically configured to operate based on static, user-selected, or preprogrammed settings, such as fixed temperature, duration, stimulus type, intensity parameters, or other operational parameters. Those parameter types and others that affect the therapeutic outcome are the therapeutic control parameters that are controlled by the controller apparatus by way of actuator components.

While static device settings may be suitable for general use, they often fail to account for individual differences in physiological response. Users may vary significantly in their tolerance to therapeutic stimuli based on factors such as physical condition, acclimatization, health status, mental or physical stress, fatigue level, hydration, adaptation to their environment, or environmental conditions. As a result, a setting that is appropriate or beneficial for one user may be ineffective or unsafe for another user, even when using the same device under similar conditions, and similarly, for the same user under different conditions.

In many conventional systems, the user is responsible for manually monitoring their own physiological condition and determining when to adjust or discontinue use of the device. Such manual monitoring may rely on subjective sensations, delayed feedback, or limited external indicators, which can be unreliable or insufficient to determine the best achievable wellness outcomes or to detect emerging physiological stress. In some cases, a user may continue device operation beyond a safe threshold due to delayed perception, distraction, or lack of awareness, thereby increasing the risk of discomfort, adverse effects, or injury.

Although wearable sensors and biomarker monitoring technologies have become more widely available, such technologies are often separate from the control mechanisms of health-related devices. In existing implementations, physiological data may be displayed to the user for informational purposes only, without directly influencing device operation. Consequently, the burden remains on the user to interpret physiological data and manually adjust device settings, limiting the effectiveness of such monitoring in preventing adverse conditions or optimizing therapeutic outcomes.

Accordingly, there exists a need for systems and methods that integrate physiological data directly into the control of health-related devices, enabling automated, responsive adjustment of device operation based on the user's physiological condition. Such integration would reduce reliance on manual intervention, improve safety, and enhance the effectiveness of therapeutic exposure by adapting device behavior in real time or near real time. The present disclosure addresses these and other shortcomings of conventional approaches by providing a physiological data-driven control system, apparatus, and method for a health-related device.

### SUMMARY OF THE INVENTION

The present disclosure relates generally to systems, apparatus, and methods, using non-transitory computer-readable storage media for controlling operation of a health-related device based on physiological data obtained from or about a user. More particularly, the disclosure provides a physiological data-driven control method and apparatus in which biomarker measurements are used to preset and adjust one or more operational characteristics of a therapeutic device during use by the user. In this disclosure, the terms biomarker and biometric are distinguished. Biomarkers are biological, medical indicators (e.g., blood pressure, skin temperature, etc.) used to assess physiological state, condition, or health status, whereas biometrics are unique, measurable physical traits (e.g., fingerprints, facial recognition) used for identification and security.

In accordance with some embodiments, a system is provided that includes at least one biomarker sensor configured to obtain physiological data associated with a user, a controller communicatively coupled to the biomarker sensors, and a health-related device operatively coupled to the controller. The controller is configured to receive physiological data from the biomarker sensors, determine at least one therapeutic control parameter value based on the physiological data, generate one or more control signals comprising electrical or digital control outputs corresponding to the therapeutic control parameter value, and cause modification of at least one operational characteristic of the health-related device in response to the control signals.

The system, apparatus, and method will have the ability to utilize “pre-measured” input data measured when the health-related device is not in use by the user, whether prior to use or after an earlier use, and/or input data based on the monitoring of biomarkers during use of the device. In this disclosure, monitoring a biomarker during the use of the device will be considered “real-time” monitoring. The controller can use either type (pre-measured or real-time) of data or combine the information of both. Operation of the controller may update the algorithm for processing future pre-measured inputs, to better establish initial settings for the particular user, and can be stored in the controller or in an associated software application, and may be saved for the next or later use.

Real-time biomarker data is received from the sensor(s) and analyzed while the device is in use, allowing the system to make adjustments to the health improvement devices in response to any detected changes in biological processes. This ensures immediate corrective actions for performance and prevents potential health crises.

Pre-measured biomarker data allows control through measurements that are made either while the user is not using the device, e.g., daily information about the user, or physical parameters of the user's body type, which in some embodiments can be height, weight, age, biomarkers measured by wearables such as a ring or a watch that can indicate physiological parameters prior to using the wellness device, or accumulated information over a period of time that helps with presetting the control outputs of the controller.

In some embodiments, the controller combines pre-measured and real-time biomarker data to adaptively refine therapeutic control parameters across sessions. In such embodiments, the controller may initiate a session using therapeutic parameter values established from pre-measured data, and during active use of the health-related device, receive real-time physiological data that informs whether those initial parameter values are producing the intended physiological response. The controller may use the observed relationship between the pre-measured inputs and the real-time physiological outcomes to update the algorithm for determining future pre-measured-based parameter values, such that initial session settings more accurately conform to the user's physiological response profile over time. In some embodiments, the pre-measured parameter values serve as a baseline against which real-time physiological data is compared, and the controller determines adjustments to the parameter determination process based on the degree to which real-time measurements deviate from expected responses to the initial parameter values.

As used herein, a therapeutic control parameter refers to any measurable or configurable variable that determines, influences, or regulates a therapeutic function or operational characteristic of a health-related device, including without limitation temperature, rate of temperature change, duration of therapy, intensity of therapy, flow rate, pressure, and power level. A therapeutic control parameter value, as used herein, refers to a specific quantitative or qualitative instance of a therapeutic control parameter determined by the controller based on received physiological data.

As used herein, a biomarker sensor refers to any device, component, or system configured to measure one or more physiological or biological parameters of a user, including without limitation wearable sensors, implantable sensors, ingestible sensors, optical sensors, infrared sensors, electrical sensors, and environmental sensors. Biomarker sensors include, without limitation, devices such as smartwatches, wearable bands, rings, chest straps, finger clips, and other physiological monitoring devices.

As used herein, a health-related device refers to any device, system, or apparatus designed or used for therapeutic, rehabilitative, wellness, or health maintenance purposes, including without limitation cold plunge systems, sauna systems, thermal therapy systems, electrical muscle stimulation devices, transcutaneous electrical nerve stimulation devices, massage devices, radiant energy devices, hyperbaric chambers, rehabilitation devices, fitness recovery devices, and wearable therapeutic devices.

As used herein, electrical or digital control outputs refer to control signals generated by the controller for the purpose of modifying one or more physical operational characteristics of the health-related device. Electrical control outputs include, without limitation, AC voltages, DC voltages, AC currents, DC currents, pulse-width modulated signals, and relay activation signals. Digital control outputs include, without limitation, digital control commands, driver-level control signals, and other actuation component instructions transmitted in digital form. The foregoing examples are illustrative and non-exhaustive, and outputs may take any electrical or digital form suitable for driving one or more actuation components of the health-related device. Such electrical or digital control outputs are received by one or more actuation components of the health-related device and cause modification of at least one operational characteristic thereof.

The system and associated method of the present disclosure overcome one or more of the shortcomings of the prior art. Additional features and advantages may be realized through the techniques of the present disclosure. Other embodiments and aspects of the disclosure are described in detail herein and are considered a part of the claimed disclosure. These and other objects, features, and advantages of this disclosure will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1A shows a block diagram of a health-related device control system in accordance with some embodiments of the present disclosure;

FIG. 1B shows a health-related device control system in accordance with some embodiments of the present disclosure;

FIG. 2A shows a detailed block diagram of a controller in accordance with some embodiments of the present disclosure;

FIG. 3 shows a flow diagram for controlling operation of a health-related device based on physiological data in accordance with some embodiments of the present disclosure;

FIG. 4A shows a schematic representation of computer-readable storage medium in accordance with some embodiments of the present disclosure;

FIG. 4B shows simplified signal-processing in which data from a single sensor is provided to a window function module in accordance with some embodiments of the present disclosure;

FIG. 4C illustrates multi-sensor processing in accordance with some embodiments of the present disclosure;

FIG. 5A shows an example health-related device embodiment configured as a sauna enclosure in accordance with some embodiments of the present disclosure;

FIG. 5B shows an embodiment of the health-related device configured as a sauna enclosure capable of delivering multiple emission wavelengths and controlled environmental circulation in accordance with some embodiments of the present disclosure;

FIG. 5C shows an example health-related device embodiment configured to provide a cold or hot therapeutic fluid environment in accordance with some embodiments of the present disclosure;

FIG. 5D shows an example system architecture for a health-related device configured to operate in conjunction with wearable biomarker sensors and a user device in accordance with some embodiments of the present disclosure;

FIG. 6 illustrates a voice-assisted control architecture integrated with the controller in accordance with some embodiments of the present disclosure; and

FIG. 7 illustrates a machine-learning-based physiological modeling architecture used by the controller in accordance with some embodiments of the present disclosure.

Like reference numerals refer to like parts throughout the several views of the drawings.

### DETAILED DESCRIPTION OF THE INVENTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

Unless the context requires otherwise, throughout the specification and claims which follow, the word “comprise” and variations thereof, such as “comprises” and “comprising” are to be construed in an open, inclusive sense, that is as “including, but not limited to.”

As used in this specification and the appended claims, the singular forms “a,” “an,” and “the” include plural referents unless the context clearly dictates otherwise.

The headings and Abstract of the Disclosure provided herein are for convenience only and do not interpret the scope or meaning of the embodiments.

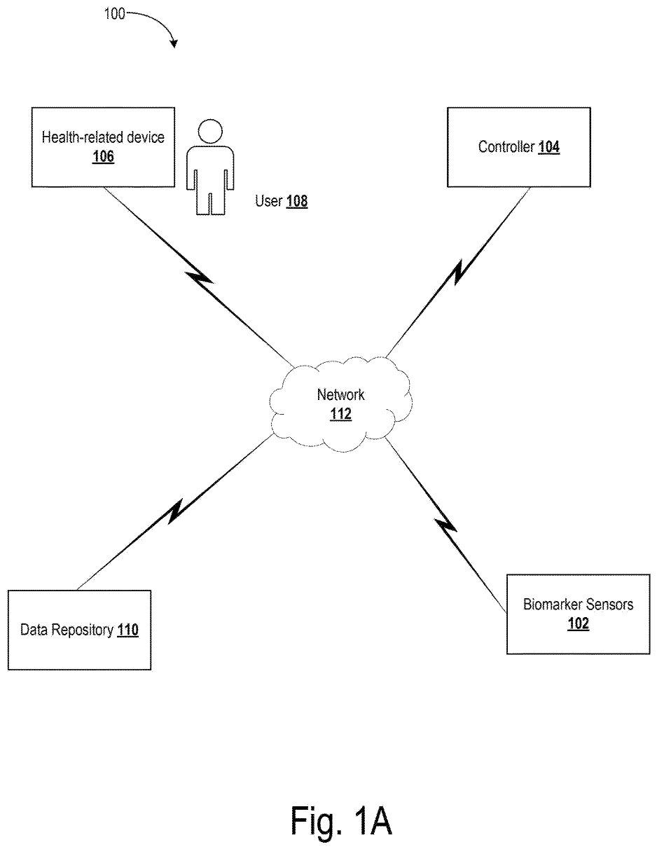

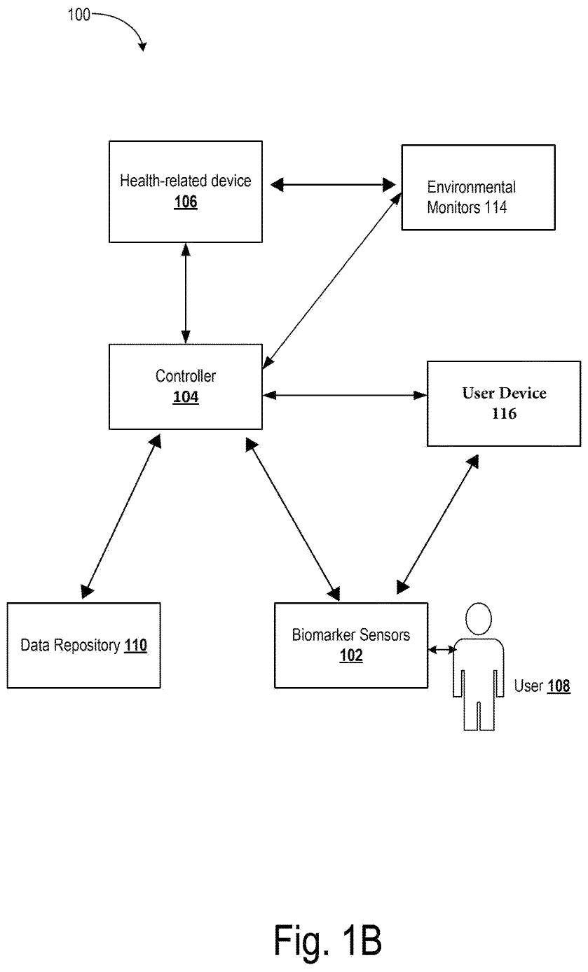

Referring initially to FIG. 1A, which illustrates an example health-related device control system 100 in accordance with some embodiments of the present disclosure. As shown in FIG. 1A, the control system 100 includes one or more biomarker sensors 102, a controller 104, a health-related device 106, a user 108, a data repository 110, and a network 112.

The biomarker sensors 102 are configured to obtain physiological data associated with the user 108. The physiological data may include real-time biomarker data, pre-measured biomarker data, or a combination thereof, and may represent physiological parameters such as temperature, cardiovascular indicators, metabolic indicators, respiratory indicators, or other biological measurements associated with the user. The data may be conveyed between the sensor(s) and the input stage of the controller by different media and format, depending on the sensor source. Each sensor may have a different type of connection to the controller. For example, if the sensor is an analog type with a wire to the controller, the receiver in the controller will have signal conditioning and possibly detection, analysis, sampling, digitizing, organizing, and formatting of the data to be stored in the controller's memory. If the sensor device has a self-contained Bluetooth capability, it can communicate with the controller and the controller can request its data or the controller can be made aware when the data is available. Similarly for a Wi-Fi based sensor, network sensor, or any wired or wireless connectivity.

The biomarker sensors 102 may include wearable, implantable, ingestible, optical, infrared-sensing, or other physiological sensing devices. Examples of commercially available biomarker sensing devices include smartwatches, wearable bands such as the Whoop Strap, chest straps such as the Polar H10, finger rings, thermal sensors such as the FLIR One, muscle oxygenation monitors such as the Moxy Monitor, and other wearable biosensors. The foregoing list is illustrative and non-exhaustive.

The controller 104 is configured to receive physiological data from the biomarker sensors 102, determine at least one therapeutic control parameter value based on the physiological data, generate one or more control signals comprising electrical or digital control outputs corresponding to the therapeutic control parameter value, and cause modification of at least one operational characteristic of the health-related device 106 in response to the control signals. The controller may evaluate the physiological data against predefined thresholds, baseline values, or user-specific physiological characteristics to determine therapeutic control parameter values suitable for maintaining the user within an acceptable therapeutic and physiological range.

In some embodiments, the controller may include a data conditioner, which receives data from multiple sensor sources typically not all in the same format, and conditions, formats, and normalizes the data, so that it can be readily processed by the controller to evaluate the therapeutic outcomes of the device. The data conditioner may perform signal normalization, outlier removal, unit conversion, and construction of a unified data structure suitable for downstream processing modules. The data conditioner may further perform adaptive baseline functions, including computation of moving averages, rolling standard deviations, and dynamic recalibration of baseline physiological reference values based on historical session data.

In some embodiments, the resulting evaluation outcomes result in control requirements that are effected by the controller through electrical or digital control outputs. Such outputs drive one or more actuation components that physically modify the operation of the health-related device. As used herein, the term “output” refers to electrical or digital control signals generated by the controller for the purpose of modifying one or more physical operational characteristics of the health-related device. Electrical control outputs include, without limitation, AC voltages, DC voltages, AC currents, DC currents, pulse-width modulated signals, and relay activation signals. Digital control outputs include, without limitation, digital control commands, driver-level control signals, and other actuation component instructions transmitted in digital form. The foregoing examples are illustrative and non-exhaustive, and outputs may take any electrical or digital form suitable for driving one or more actuation components of the health-related device.

In some embodiments, outputs generated by the controller ultimately affect physical elements of the health-related device, including without limitation pumps, valves, heating elements, cooling elements, fans, compressors, electromagnetic radiators, flow guides, ice generation mechanisms, acoustic emitters, vibration sources, pressure-modulating devices, and relays configured to selectively connect an electrical source to a load within the health-related device. For purposes of this disclosure, an actuation component includes the control of anything that changes the operational behavior of the health-related device, including drivers, switches, relays, emitters, or any control effector.

The health-related device 106 comprises a physical device configured to provide health maintenance or health improvement benefits to the user 108. In some embodiments, the health-related device 106 comprises a therapeutic device, a temperature control subsystem, and one or more actuation components. The health-related device may comprise, without limitation, a cold plunge system, a sauna system, a thermal therapy system, an electrical muscle stimulation device, a transcutaneous electrical nerve stimulation device, a massage device, a radiant energy device, a hyperbaric chamber, a rehabilitation device, a fitness recovery device, or a wearable therapeutic device.

The data repository 110 may be implemented as an on-device storage system, a cloud-based storage system, or a distributed data storage architecture. The data repository may also be used as intermediate storage to update firmware or software programs and save older versions for reversion and version control.

The network 112 may include any suitable communication infrastructure enabling data exchange among the biomarker sensors 102, the controller 104, the health-related device 106, and the data repository 110. By way of example, the network 112 may include a local area network (LAN), a wide area network (WAN), a cellular network, the Internet, or a combination thereof, including star networks, ring networks, hybrid networks, and combinations of simplex, duplex, and full-duplex connections. In some embodiments, secure communication protocols may be employed to protect physiological data and control signals transmitted over the network 112.

In some embodiments, transmission of physiological data from biomarker sensors to the controller may occur via wired or wireless communication, including direct connections, network-based paths such as the Internet, a network bridge, a router, or other network infrastructure, and combinations thereof. Such transmission flexibility applies regardless of whether the physiological data comprises real-time biomarker data received during use of the health-related device, pre-measured biomarker data obtained when the health-related device is not in use by the user, or a combination of both.

Although FIG. 1A illustrates a single controller 104, a single health-related device 106, a single data repository 110, and a single set of biomarker sensors 102, it will be appreciated that multiple instances of any of these components may be deployed in distributed or scalable implementations. Further, although the biomarker sensors 102, the controller 104, and the health-related device 106 are illustrated as separate components, in some embodiments two or more of these components may be integrated into a single unit or device without departing from the scope of the present disclosure.

FIG. 1B illustrates an embodiment of the health-related device control system 100 in accordance with some embodiments of the present disclosure. In FIG. 1B, one or more components of the system may be integrated within or physically associated with the health-related device 106, forming a partially or fully integrated control architecture.

As shown in FIG. 1B, the health-related device 106 may include the controller 104, one or more biomarker sensors 102, a data repository 110, a user device 116 such as a phone or tablet, and one or more environmental monitors 114. In this embodiment, the controller 104 is disposed within or directly coupled to the health-related device 106 and may be configured to receive physiological data from the biomarker sensors 102 associated with the user 108.

The environmental monitors 114 represent one or more sensors configured to monitor operational and environmental parameters of the health-related device 106. In some embodiments, the environmental monitors 114 may measure temperature of a therapeutic medium, ambient temperature, humidity, fluid level, pressure, flow rate, light wavelength or intensity, electrical current, voltage, motor speed, or other parameters associated with operation of the health-related device 106. The environmental monitors 114 provide device-condition data to the controller 104, enabling the controller to account for both user physiological state and device operating conditions when determining therapeutic control parameter values.

In some embodiments, the controller 104 or user device 116 processes physiological data received from the biomarker sensors 102 together with environmental data received from the environmental monitors 114 to generate structured data representations for control analysis. By integrating user-specific physiological inputs and device-specific environmental measurements, the controller 104 may determine whether modifications to operational characteristics of the health-related device 106 are required to maintain operation within acceptable therapeutic and safety ranges.

The data repository 110, which may be integrated within the health-related device 106 as shown, is configured to store physiological data, baseline values, prior session data, environmental history data, firmware versions, and control parameters. In some embodiments, the data repository 110 supports retrieval of historical physiological and environmental information to improve subsequent control decisions and adaptive baseline adjustments.

In the embodiment illustrated in FIG. 1B, a user device 116 may communicate with the health-related device 106. The user device 116 may provide user inputs, configuration preferences, or display device status information through an application. In some embodiments, the user device may operate an application that collects data from the biomarker sensors, operates on that data, and then communicates the operated-on data to the controller. In some embodiments, the user device may determine the most appropriate control function for the health-related device and communicate that information to the controller, which then operates the health-related device directly. In some embodiments, the user device 116 is optional, and the controller 104 may operate autonomously without reliance on external user interaction.

Operation of the system of FIG. 1B may occur as a closed-loop control process in which physiological data from the biomarker sensors 102 and environmental data from the environmental monitors 114 are continuously, periodically, aperiodically, or on-demand provided to the controller 104. The controller 104 evaluates the combined data and generates control signals to modify one or more operational characteristics of the health-related device 106, such as temperature, circulation rate, duration, intensity, or rate of change. By monitoring both user condition and device condition, the system provides coordinated, responsive, and safety-aware operation of the health-related device.

Although FIG. 1B illustrates the controller 104, biomarker sensors 102, data repository 110, and environmental monitors 114 as integrated within the health-related device 106, it will be appreciated that such components may alternatively be distributed, partially integrated, or externally located, without departing from the scope of the present disclosure.

In some embodiments, the health-related device 106 may be a sauna system, and the controller 104 includes ON/OFF control circuitry, a temperature detection subsystem, and an external wall-mounted control panel positioned outside the sauna. The external control panel may serve as an installation point for user configuration and monitoring while maintaining separation from the internal thermal environment of the sauna.

In some embodiments, temperature sensing within the sauna is performed using a thermistor configured to generate an analog voltage proportional to measured temperature. The thermistor output may be provided to the controller 104, where the analog signal is converted to a digital representation by an analog-to-digital conversion (ADC) stage. In some embodiments, the ADC provides an 8-bit digital output corresponding to the measured temperature value. The digitized temperature signal may then be transmitted to a processor within the controller for evaluation and control determination.

In some embodiments, the controller 104 is not physically integrated within the health-related device 106 and may instead be remotely located, partially integrated, or distributed across multiple devices. For example, the controller 104 may be implemented within a separate control enclosure, a wall-mounted control unit positioned outside a sauna, a user device, or in part in a cloud-connected processing system. In some embodiments, the remote part of the controller 104 remains communicatively coupled to the actuators of the health-related device 106 via wired or wireless communication links and performs control determination independent of physical placement.

In some embodiments, the biomarker sensors 102 comprise one or more wearable devices positioned on the user 108 and located outside the therapeutic device during at least a portion of operation. For example, the biomarker sensors 102 may include a smartwatch, wearable band, ring, chest strap, ring for a finger, finger clip, or other wearable monitoring device configured to obtain physiological data such as heart rate, heart rate variability, skin temperature, or stress-related indicators. The biomarker sensors 102 may communicate physiological data to the controller 104 directly or indirectly via the network 112.

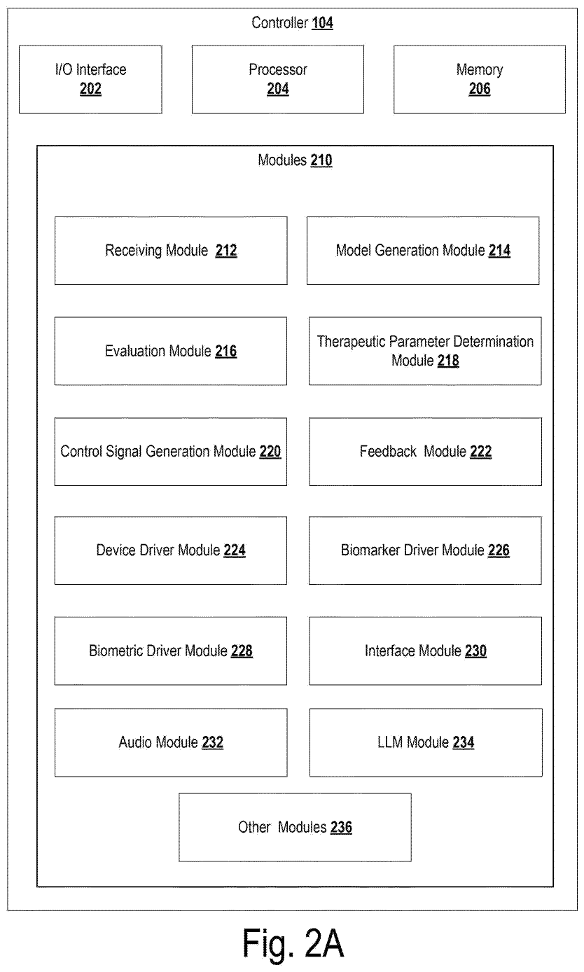

FIG. 2A shows a detailed block diagram of the controller 104 in accordance with some embodiments of the present disclosure.

In some embodiments, the controller 104 includes an I/O interface 202, a processor 204, a memory 206, and one or more modules 210. In some embodiments, modules may be non-transitory computer readable storage media called by the main routine of the processor to implement specific functions. The I/O interface 202 is configured to communicate with the biomarker sensors 102, the health-related device 106, and the data repository 110 via the network 112. The processor 204 is configured to execute instructions to receive physiological data associated with a user, determine physiological conditions of the user, determine therapeutic control parameter values, and generate control signals for controlling operation of the health-related device 106. The memory 206 stores executable instructions, configuration parameters, physiological thresholds, user-specific baseline data, pre-measured biomarker data, and runtime data utilized by the modules 210.

In some embodiments, the controller 104 includes the modules 210 for performing the various operations described herein. The modules 210 may be implemented as software instructions executed by the processor 204, as firmware, as dedicated hardware logic, or as any combination thereof. The modules 210 may include, without limitation, a Receiving Module 212, a Model Generation Module 214, an Evaluation Module 216, a Therapeutic Parameter Determination Module 218, a Control Signal Generation Module 220, a Feedback Module 222, and one or more Other Modules 236. As used herein, the term “module” refers to hardware, firmware, software, or any combination thereof capable of performing the associated functionality.

The Receiving Module 212 is configured to receive physiological data associated with a user from the biomarker sensors 102. The physiological data may include real-time biomarker data, pre-measured biomarker data obtained when the health-related device 106 is not in use, or a combination thereof. In some embodiments, the Receiving Module 212 is operable to format, timestamp, validate, or pair received physiological data with a particular user or session prior to further processing. In some embodiments, the Receiving Module 212 performs data conditioning operations on the received physiological data, including signal normalization, outlier removal, unit conversion, and construction of a unified data structure suitable for downstream processing modules. The Receiving Module 212 may further perform adaptive baseline functions, including computation of moving averages, rolling standard deviations, and dynamic recalibration of baseline physiological reference values based on historical session data.

The Model Generation Module 214 is configured to construct, maintain, and update a physiological and therapeutic model corresponding to the user. The model may contain individualized physiological response characteristics of the user to therapeutic stimuli and may be used to determine therapeutic control parameter values. The model may be initialized using pre-measured physiological data and refined during subsequent operation of the health-related device based on observed physiological responses. The model may represent the user's physiological responses using one or more data structures, statistical representations, or computational models. In some embodiments, the data structure comprises computational parameters, including model weights, feature coefficients, classification thresholds, and other learned or configured values, that are operable to determine therapeutic control parameter values from received physiological data. The model may be saved in memory specific to a user and be accessible by a non-secure interface such as a simple control knob, selection, touchpad, or button selection, or through a secure means such as passkey, password input, two-factor authentication, biometric input including optical, tactile, and audio methods of verification, or through the use of an application operating on a separate device such as a phone.

In some embodiments, the physiological and therapeutic model generated by the Model Generation Module 214 is further configured to predict user physiological responses to candidate therapeutic control parameter values and to constrain the therapeutic control parameter determination process to physiologically appropriate ranges based on the predicted responses. The model may apply temporal weighting and decay functions to historical physiological measurements such that more recent measurements receive proportionally greater influence on model updates than older measurements. In some embodiments, the user-specific model is stored in memory as a user-specific profile accessible through secure authentication, ensuring that physiological and therapeutic profiles are isolated per user.

The Evaluation Module 216 is configured to analyze physiological data received from the Receiving Module 212 and evaluate it against predefined thresholds, baseline values, or user-specific physiological characteristics. The Evaluation Module 216 may determine whether the physiological data indicates that the user is within an acceptable therapeutic and physiological range, or whether adjustment of therapeutic control parameter values is required. The Evaluation Module 216 may generate one or more evaluation outputs indicative of physiological stress level, safety condition status, or recommended parameter adjustments. The Evaluation Module 216 may further detect physiological safety conditions including hypothermia risk, hyperthermia risk, oxygen desaturation, cardiovascular stress, or neuromuscular fatigue, and initiate corrective control actions or safety notifications in response.

The Therapeutic Parameter Determination Module 218 is configured to determine one or more therapeutic control parameter values based on the physiological data received from the Receiving Module 212 and the physiological and therapeutic model generated by the Model Generation Module 214. The Therapeutic Parameter Determination Module 218 may execute rule-based logic, statistical analysis, machine learning, or artificial intelligence algorithms to determine appropriate therapeutic control parameter values. In some embodiments, the Therapeutic Parameter Determination Module 218 maintains a therapeutic parameter range associated with each therapeutic control parameter value and constrains determined values within the therapeutic parameter range during device operation. Safety-critical constraints enforced by the Therapeutic Parameter Determination Module 218 remain authoritative and may not be overridden by machine learning outputs or conversational inputs. In some embodiments, physiological data, voice input, model outputs, and other input data are each provided as an input to the determination of the at least one therapeutic control parameter value by the Therapeutic Parameter Determination Module 218, such that the determination is performed using one or more of such inputs.

The Control Signal Generation Module 220 is configured to generate one or more control signals corresponding to the therapeutic control parameter values determined by the Therapeutic Parameter Determination Module 218. The control signals comprise electrical or digital control outputs configured to drive one or more actuation components of the health-related device to modify at least one operational characteristic thereof. In some embodiments, the Control Signal Generation Module 220 translates therapeutic control parameter values into device-compatible signals, which may include digital control commands, pulse-width modulated signals, relay activation signals, voltage or current outputs, or other hardware-compatible control outputs.

The Feedback Module 222 is configured to receive updated physiological data from the biomarker sensors 102 following modification of operational characteristics of the health-related device 106, and to provide the updated physiological data to the Receiving Module 212 for continued evaluation. In some embodiments, the Feedback Module 222 enables closed-loop operation of the controller 104 by continuously, periodically, aperiodically, or on-demand supplying updated physiological measurements for use in further therapeutic parameter determination. As used herein, a closed-loop control process refers to a control architecture in which updated input data is received by the controller following modification of operational characteristics of the health-related device, and the controller uses the updated input data to determine revised therapeutic control parameter values and generate revised control signals, such that the output of the controlled system feeds back into the control determination process.

In some embodiments, the controller 104 further includes one or more driver modules configured to interface directly with the hardware components of the health-related device 106. Driver modules may include a Device Driver Module 224, a Biomarker Driver Module 226, and a Biometric Driver Module 228. The Device Driver Module 224 is configured to convert control outputs generated by the Control Signal Generation Module 220 into hardware-specific commands compatible with the health-related device 106. The Device Driver Module 224 performs driver-level hardware conversion, translating therapeutic parameter values into electrical signals such as voltages, currents, pulse-width modulated waveforms, relay switching commands, or other hardware-compatible control outputs required by the actuation components of the health-related device. In some embodiments, driver-level hardware conversion includes conversion of digital signals to analog electrical signals (digital-to-analog conversion (DAC)), enabling the controller to drive actuation components that require analog input signals. The Biomarker Driver Module 226 is configured to interface with biomarker sensors 102 and manage acquisition, formatting, buffering, and transmission of physiological measurements.

In some embodiments, the Biometric Driver Module 228 is configured to perform user authentication using biometric identifiers, including but not limited to heart rate variability signatures, cardiovascular response patterns, voice signatures, fingerprint data, facial recognition data, or other biometric markers. The Biometric Driver Module 228 may verify that physiological data being processed by the controller 104 corresponds to an authorized user, thereby preventing unauthorized individuals from influencing device operation or accessing stored physiological data.

In some embodiments, the controller 104 further includes an Interface Module 230 and an Audio Module 232. The Interface Module 230 may provide graphical user interface functionality, communication with a user device, session configuration management, and status display operations. The Audio Module 232 may support voice command reception including noise mitigation and voice activation, audio prompts, or acoustic feedback to the user. In some embodiments, the Audio Module 232 interfaces with a Large Language Model (LLM) Module 234 configured to interpret user voice inputs or generate contextual feedback.

The Large Language Model (LLM) Module 234, when present, may assist in interpreting conversational input, session requests, or contextual instructions provided by the user. However, in some embodiments, outputs from the LLM Module 234 are constrained by deterministic safety logic within the Therapeutic Parameter Determination Module 218 or a dedicated safety enforcement module. Accordingly, conversational or interpretive outputs generated by the LLM Module 234 do not override predefined physiological thresholds, safety constraints, or device operating limits.

In some embodiments, the controller 104 is configured to associate physiological data, biometric identifiers, and therapeutic models with a specific authenticated user profile stored in memory 206 or in a secure data repository. Access to stored physiological and biometric data may be restricted using encryption, authentication protocols, user-specific keys, or secure enclave processing. In some embodiments, the controller 104 verifies user identity prior to enabling therapeutic operation of the health-related device 106 using at least one of biometric identification data, a passkey, a password, two-factor authentication, or a voice biometric signature.

In some embodiments, the controller includes a voice interface configured to receive spoken input from the user. The voice input may be processed by a speech recognition engine and further interpreted using a large language model (LLM) to determine user intent, preferences, or requested therapeutic adjustments. The LLM may assist in contextual interpretation and generation of speech-based responses; however, safety-critical control decisions remain subject to deterministic validation and predefined physiological thresholds. In certain embodiments, voice characteristics such as vocal patterns or biometric voice signatures may be used for user authentication, and voice-derived inputs may be combined with physiological biomarker data and other user inputs when determining therapeutic control parameter values.

The Other Modules 236 may include additional functional components that support operation of the controller 104. Such modules may include safety enforcement modules, alert generation modules, device compatibility modules, data logging modules, or system diagnostics modules. It will be appreciated that the modules illustrated in FIG. 2A may be combined, fully integrated, omitted, or reconfigured without departing from the scope of the present disclosure.

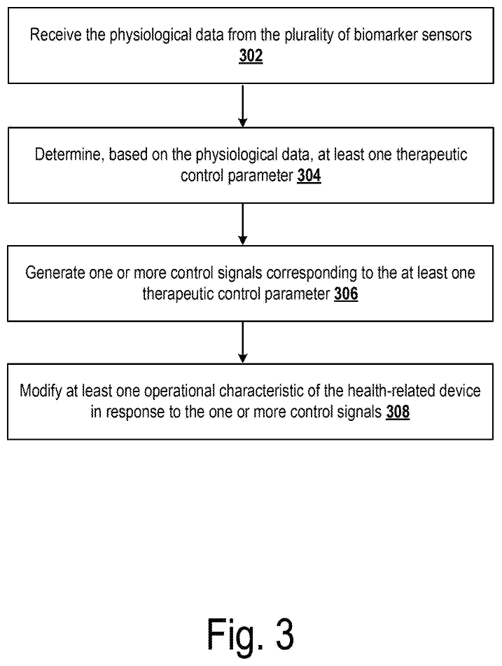

FIG. 3 illustrates an example method 300 for controlling operation of a health-related device based on physiological data in accordance with some embodiments of the present disclosure. Although method 300 is illustrated as a sequence of operations, it will be appreciated that one or more of the operations may be performed in parallel, in a different order, or with additional or fewer operations without departing from the scope of the present disclosure.

At operation 302, the system receives physiological data from at least one biomarker sensor. The physiological data may include real-time biomarker data, pre-measured biomarker data, or a combination thereof. The physiological data may include, without limitation, body temperature, skin temperature, heart rate, heart rate variability, blood oxygen saturation, respiratory rate, metabolic indicators, or other physiological measurements associated with a user. In some embodiments, physiological data may further include neurological activity measurements, such as electroencephalographic (EEG) signals or nerve conduction indicators, and neuromuscular activity measurements, such as electromyographic (EMG) signals or muscle activation patterns, obtained from appropriate biosensors configured to measure such parameters.

At operation 304, the system determines, based on the physiological data received at operation 302, at least one therapeutic control parameter value for the health-related device. The determination may include evaluating the physiological data against predefined thresholds, baseline values, safety limits, or user-specific physiological characteristics. The therapeutic control parameter value may correspond to temperature, duration, intensity, rate of change, flow rate, pressure, or power level.

At operation 306, the system generates one or more control signals comprising electrical or digital control outputs corresponding to the at least one therapeutic control parameter value determined at operation 304. The control signals are configured to be compatible with the control interface of the health-related device and may include digital commands, pulse-width modulated signals, relay activation signals, or other electrical or digital control outputs suitable for modifying operation of the device.

At operation 308, the system modifies at least one operational characteristic of the health-related device in response to the one or more control signals generated at operation 306. Modification of the operational characteristic may occur automatically and during active use of the device by the user. In some embodiments, operations 302 through 308 are repeated continuously, periodically, aperiodically, or on-demand to form a closed-loop control process, wherein updated physiological data is used to further adjust operation of the health-related device over time.

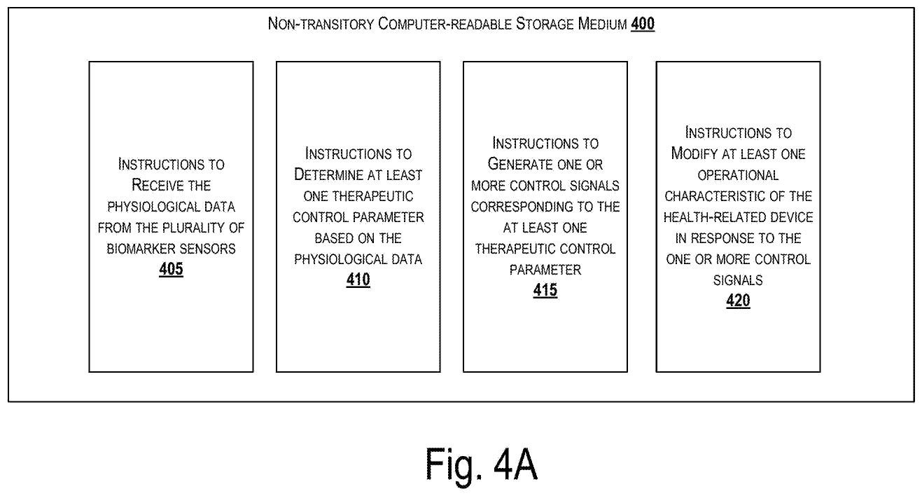

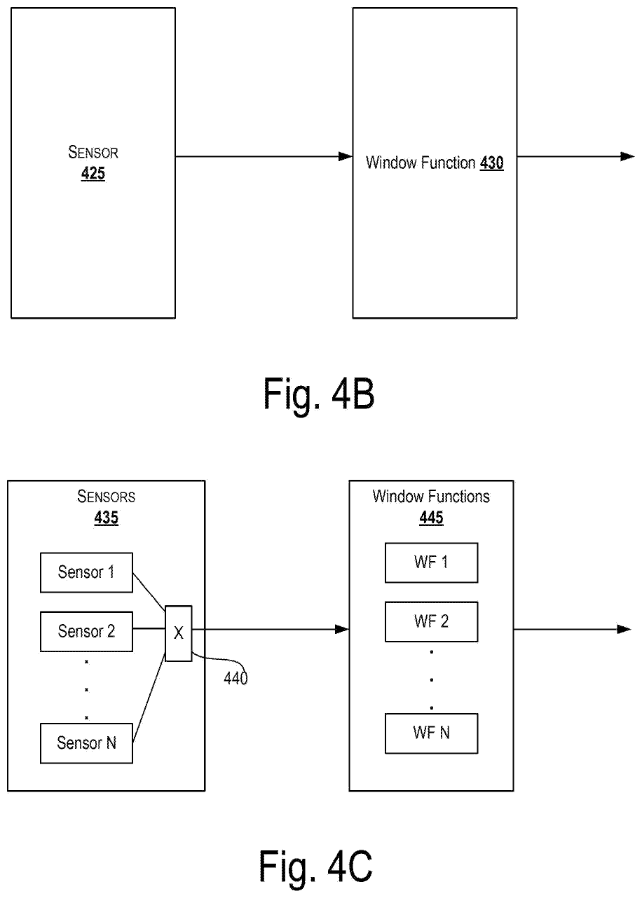

FIG. 4A illustrates an example non-transitory computer-readable storage medium 400 in accordance with some embodiments of the present disclosure. The non-transitory computer-readable storage medium 400 stores instructions that, when executed by one or more processors of a controller, cause the controller to perform operations for controlling operation of a health-related device based on physiological data obtained from a user. The instructions stored on the medium include instructions 405 configured to cause receipt of physiological data from at least one biomarker sensor; instructions 410 configured to cause determination of at least one therapeutic control parameter value based on the physiological data; instructions 415 configured to cause generation of one or more control signals comprising electrical or digital control outputs corresponding to the at least one therapeutic control parameter value; and instructions 420 configured to cause modification of at least one operational characteristic of the health-related device in response to the one or more control signals.

In some embodiments, execution of the instructions 405, 410, 415, and 420 causes the controller to perform the operations of method 300 described with respect to FIG. 3. The instructions stored on the non-transitory computer-readable storage medium 400 may be executed continuously, periodically, aperiodically, or on-demand to enable closed-loop control of the health-related device based on updated physiological data.

The methods described herein may be performed using the systems described herein. Moreover, the systems described herein may perform the methods described herein and may perform or execute the instructions stored in the computer readable storage media described herein. It is also contemplated that the systems described herein may perform functions or execute instructions other than those described in relation to the methods and computer-readable storage media described herein.

FIG. 4B illustrates a simplified signal-processing embodiment in which data from a single sensor 425 is provided to a window function module 430. In this embodiment, the sensor 425 may represent any physiological or environmental sensing element configured to generate time-varying data. The window function module 430 applies a selected windowing operation to the received sensor data prior to further processing. The windowing operation may include, without limitation, rectangular windows, Kaiser windows, Hamming windows, Hanning windows, or other digital windowing functions used to shape, filter, segment, or weight time-domain data. The window function 430 may operate on fixed-length segments, sliding windows, adaptive windows, or dynamically sized intervals based on signal characteristics.

FIG. 4C illustrates a multi-sensor processing embodiment in which a plurality of sensors 435, including Sensor 1 through Sensor N, provide data to a combiner 440. The combiner 440 may apply weighted aggregation, scaling, normalization, or other signal combination techniques to produce a composite signal. In some embodiments, individual window functions 445 (WF1 through WFN) may be applied to respective sensor inputs prior to combination. In other embodiments, window functions may be applied after weighted combination of the sensor signals. In some embodiments, the combiner 440 may implement weighted summation, adaptive weighting, correlation-based fusion, confidence-based weighting, or other multi-sensor fusion techniques. The weighting factors may be predetermined, dynamically adjusted based on signal quality, signal-to-noise ratio, variance, or reliability metrics, or adapted over time using algorithmic learning techniques.

In some embodiments, the sensor inputs provided by the plurality of sensors 435 are filtered prior to, during, or after combination within the combiner 440. Filtering may include moving average windowing, sliding window processing, weighted averaging, finite impulse response (FIR) filtering, infinite impulse response (IIR) filtering, or combinations thereof. The window functions 445 may be implemented as moving or sliding windows having defined durations, sample counts, or adaptive intervals, and may be applied independently to individual sensor signals or coordinated across multiple sensor inputs.

In some embodiments, filtering operations are characterized by configurable parameters including averaging window duration, sampling rate, window overlap, and associated frequency response characteristics. In some embodiments, the selection of window duration and filter parameters establishes a tradeoff between responsiveness and stability in control decisions. Larger window durations may attenuate high-frequency noise components and provide smoother composite signals, thereby improving stability in therapeutic parameter determination. Conversely, smaller window durations may increase effective bandwidth, allowing faster reaction to transient physiological variations.

In some embodiments, the controller uses on-demand sensor update requests, requesting updated physiological measurements from one or more biomarker sensors at intervals determined by the closed-loop control requirements of the system. In some embodiments, a machine learning process may adaptively modify the signal processing pipeline, including window selection, filter parameters, and sensor fusion weighting, based on observed physiological response patterns and control performance metrics. Such adaptive modification may improve the reliability and responsiveness of physiological data processing over time.

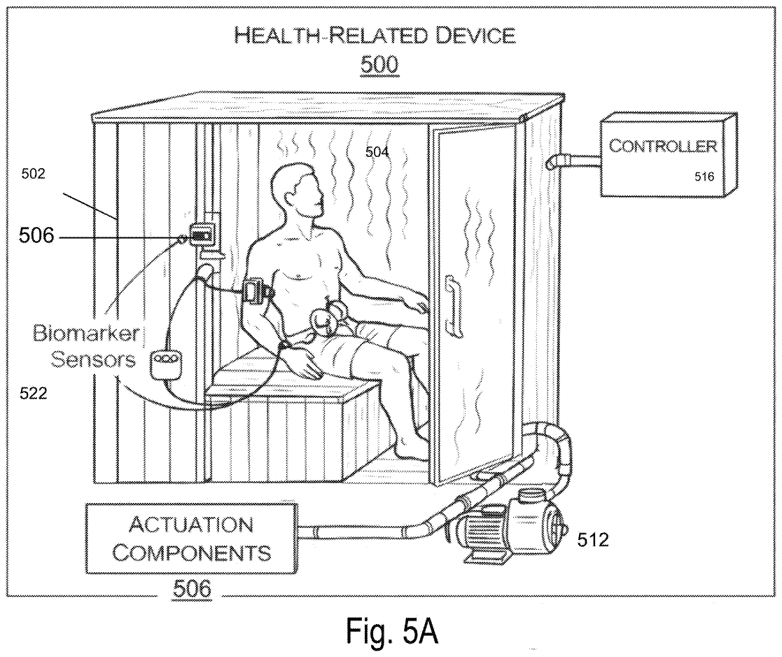

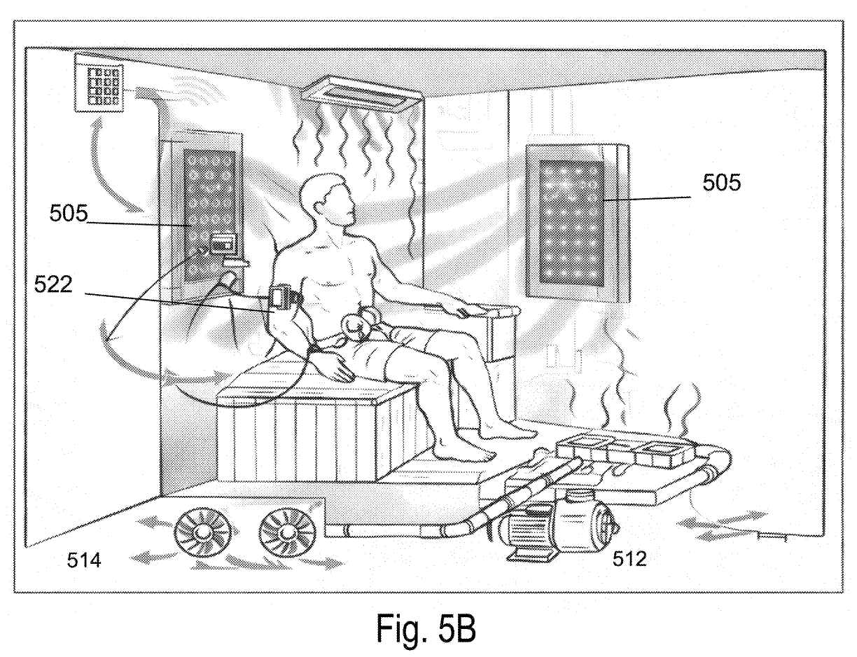

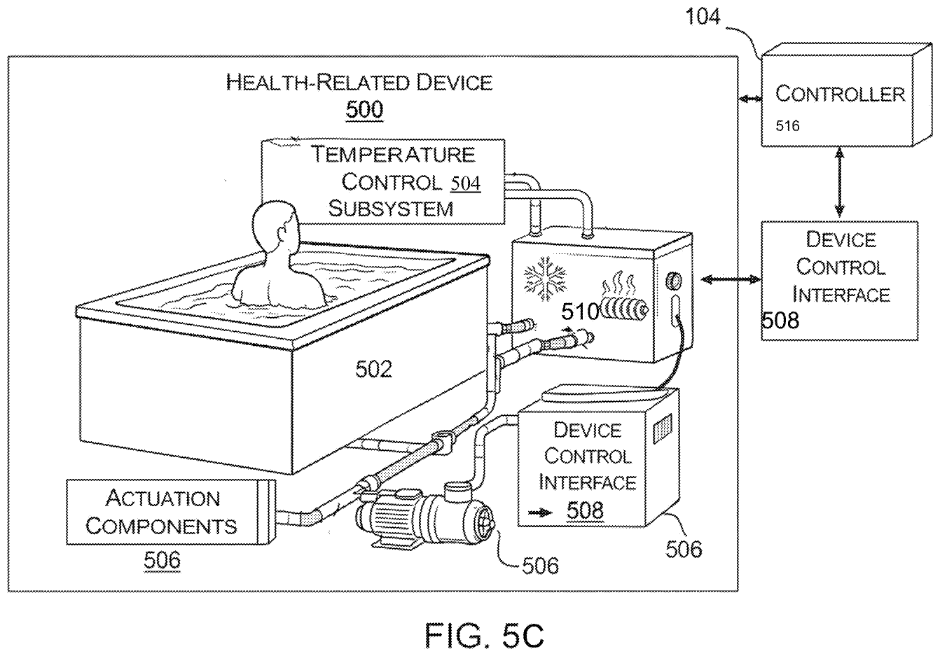

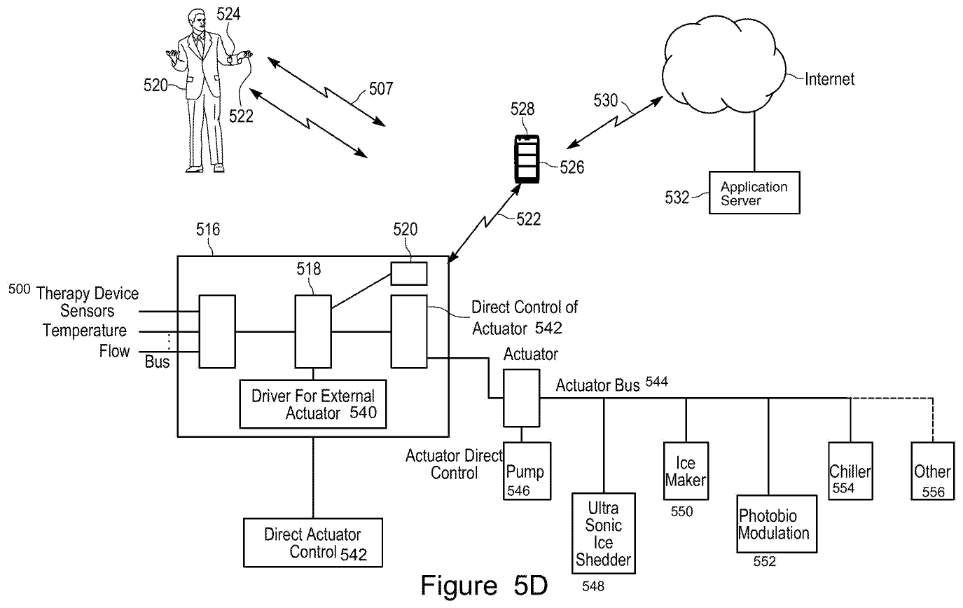

FIG. 5A illustrates an example health-related device embodiment 500 in accordance with some embodiments of the present disclosure. In the illustrated embodiment, the health-related device 500 comprises a therapeutic device 502 configured to receive at least a portion of a user's body and to provide a controlled therapeutic environment. In the embodiment shown, the therapeutic device 502 is configured as a sauna enclosure; however, it will be appreciated that other therapeutic configurations may be implemented without departing from the scope of the present disclosure.

As shown in FIG. 5A, the health-related device 500 further includes one or more actuation components 506 configured to generate, regulate, and modulate the therapeutic environment within the therapeutic device 502. The actuation components 506 may include heating elements, air circulation components, pumps 512, valves, compressors, motors, light sources, acoustic emitters, vibration sources, pressure-modulating devices, or other electronic, mechanical, or electromechanical elements configured to control temperature, airflow, humidity, emission intensity, wavelength, circulation, phase change, or other environmental parameters.

The actuation components 506 may further include pumps 512, valves, motors, circulation fans 514, compressors, lights, LEDs, sound emitters, ultrasound sources, pressure-modulating devices, or other electronic, mechanical, or electromechanical components configured to control flow rate, circulation pattern, pressure, vibration, phase change, or intensity of the therapeutic stimulus.

The health-related device 500 further includes a device control interface 508 communicatively coupled to a controller 516. The device control interface 508 is configured to receive one or more control signals generated by the controller 516 and to translate such control signals into actuation commands for the temperature control subsystem 504 and the actuation components 506. The controller 516 may be located remotely from or integrated with the health-related device 500 and may communicate with the device control interface 508 via a communication link.

The embodiment of FIG. 5A further illustrates one or more biomarker sensors 522, 524 operatively associated with a user 520 positioned within the therapeutic device 502.

The biomarker sensors 522, 524 may include wearable sensors, skin-contact sensors, optical sensors, infrared sensors, or other physiological monitoring devices configured to obtain physiological measurements such as heart rate, heart rate variability, skin temperature, oxygen saturation, or other biomarkers.

The controller 516 is configured to receive physiological data from the biomarker sensors 522, 524 and to evaluate the received data in accordance with the control methodologies described with respect to FIGS. 1-4. Using the evaluated physiological condition of the user 520 as an input, the controller 516 generates one or more control signals that are transmitted to the actuation components 506 to modify one or more operational characteristics of the therapeutic device 502.

In operation, physiological data associated with the user 520 is monitored during active use of the therapeutic device 502. The controller 516 determines whether adjustment of the therapeutic environment is required in accordance with predefined safety thresholds, baseline values, or adaptive control parameters. For example, if physiological indicators exceed configured limits, the controller 516 may cause reduction of thermal intensity, modification of airflow, or adjustment of session duration to maintain operation within acceptable physiological ranges.

Although FIG. 5A illustrates a sauna embodiment, the health-related device 500 may alternatively comprise other therapeutic devices having controllable operational characteristics. The components illustrated are exemplary, and additional components may be included, or illustrated components may be omitted or rearranged, without departing from the scope of the present disclosure.

FIG. 5B illustrates an embodiment of the health-related device 500 configured as a sauna enclosure capable of delivering multiple emission wavelengths and controlled environmental circulation. In the illustrated embodiment, the sauna includes a plurality of emission panels 505 disposed along interior surfaces of the chamber and configured to emit therapeutic radiation across multiple wavelength ranges. Such emission sources may include infrared emission elements and visible-spectrum light sources configured for light-based therapy. The sauna further includes circulation fans 514 configured to regulate airflow and environmental circulation within the chamber. In some embodiments, physiological data obtained from biomarker sensors 522, 524 associated with the user 520 is provided to the controller 516, which adjusts emission output and airflow characteristics to maintain desired therapeutic conditions and ensure operation within predefined safety thresholds.

FIG. 5C illustrates another example health-related device embodiment 500 comprising a therapeutic device 502 configured to receive at least a portion of a user's body and to provide a cold or hot therapeutic fluid environment.

As shown in FIG. 5C, the health-related device 500 further includes a temperature control subsystem 504 operatively coupled to the therapeutic device 502. The temperature control subsystem 504 may include heating elements, cooling elements, chillers 510, heat exchangers, or combinations thereof, and is configured to adjust and maintain a temperature of a therapeutic medium within the therapeutic device 502.

The actuation components 506 of the embodiment of FIG. 5C may include pumps 512, valves, motors, fans 514, compressors, or other mechanical or electromechanical components configured to control flow rate, circulation pattern, pressure, or intensity of the therapeutic stimulus.

The health-related device 500 of FIG. 5C further includes a device control interface 508 communicatively coupled to a controller 516. The device control interface 508 is configured to receive one or more control signals generated by the controller 516 and to translate such control signals into actuation commands for the temperature control subsystem 504 and the actuation components 506. The controller 516 may be located remotely from or integrated with the health-related device 500 and may communicate with the device control interface 508 via a communication link.

FIG. 5D illustrates an example system architecture for a health-related device 500 configured to operate in conjunction with wearable biomarker sensors and a user device. In the illustrated embodiment, a user 520 is associated with one or more wearable biomarker sensors, such as a wearable ring sensor 522 and a wearable watch sensor 524; however, the sensors may be non-wearable and may be remote sensors as well. These sensors are configured to measure physiological parameters associated with the user, including heart rate, heart rate variability, skin temperature, activity level, metabolic indicators, or other biomarker measurements. The sensors communicate with a user device 526 through a wireless connection 528, such as Bluetooth®, Wi-Fi, LTE, or another communication interface.

The user device 526, which may comprise a mobile phone, tablet, or other computing device, executes an application 528 configured to receive physiological data from the sensors and to provide user interaction capabilities. The application may display physiological measurements, allow user configuration of therapeutic sessions, and transmit commands or physiological information to external computing resources. In some embodiments, the user device 526 communicates with an application server 532 or remote data repository through a communication connection 530. The remote system may store session data, perform additional analytics, or provide artificial intelligence-assisted recommendations related to operation of the health-related device.

The health-related device 500, which may include a cold plunge device or other therapeutic device, includes a controller 516 configured to receive control inputs from the user device 526 through a communication connection 530. The controller 516 may also receive information directly from device sensors 536 associated with the therapeutic device, such as temperature sensors, water level sensors, pressure sensors, or flow sensors. These device sensors provide environmental and operational measurements that enable the controller 516 to determine appropriate operating conditions for the therapeutic device.

In some embodiments, the controller 516 communicates with one or more actuators through a driver for external actuator 540, a direct actuator control module 542, and an actuator bus 544. The actuators may control components of the health-related device 500, including a pump 546, ice maker 550, chiller 552, ultrasonic ice shredder 548, airflow modulators, or other therapeutic actuator devices 554 configured to regulate the therapeutic environment. The controller 516 may generate control signals that directly drive the actuators or may communicate with actuator drivers that translate digital control instructions into electrical control signals suitable for operating mechanical or electromechanical components.

In some embodiments, a first processing component of the controller is implemented on a general-purpose computing device such as a mobile phone, tablet, or laptop computer, and a second processing component is implemented on a hardware execution module directly coupled to the health-related device. The hardware execution module is configured to receive control information from the first processing component and to convert that control information into hardware-compatible electrical or digital control outputs for driving the actuation components of the health-related device. In some embodiments, at least one of the receiving, determining, or generating operations may be performed by the first processing component, and the hardware execution module may additionally perform independent processing operations.

In some embodiments, voice processing, LLM interpretation, therapeutic parameter determination, and safety validation functions may each be performed by the first processing component, the second processing component, or a combination of both, provided that safety validation is performed by at least one of the components before hardware control outputs are generated. In some embodiments, machine learning model components including feature extraction, inference, model storage, and adaptive updating may each be performed by the first processing component, the second processing component, or a combination of both, and the model may be accessible to both components through a communication interface.

In some embodiments, the first processing component, implemented on a general-purpose computing device such as a mobile phone, tablet, or laptop computer, may host all controller intelligence, including receiving input data, constructing and maintaining the physiological and therapeutic model, performing therapeutic parameter determination, executing machine learning operations, performing safety validation, and generating control instructions, while the hardware execution module functions as a dumb actuator that receives control instructions from the first processing component and converts them into hardware-compatible electrical or digital control outputs for driving the actuation components of the health-related device without performing independent therapeutic determinations. In such embodiments, the hardware execution module may comprise a microcontroller, embedded processor, relay driver, or other hardware interface device configured solely to execute received instructions and produce the corresponding electrical or digital outputs.

In some embodiments, the controller 104 is distributed across multiple devices such that the first processing component of the controller is implemented on a general-purpose computing device, such as a mobile phone, tablet, or other computing device, that communicates with one or more biomarker sensors through a wireless communication interface such as Bluetooth, Bluetooth Low Energy, Wi-Fi, LTE, or proprietary wireless technology. The first processing component may receive physiological data directly from the biomarker sensors and may perform one or more processing operations on the received physiological data, including data formatting, aggregation, normalization, filtering, baseline computation, or physiological model inference, before transmitting processed data or resulting control instructions to the second processing component implemented on the hardware execution module. In some embodiments, the first processing component hosts all controller intelligence and performs all therapeutic parameter determination, with the hardware execution module receiving the resulting control instructions and generating the corresponding hardware-compatible outputs. In some embodiments, the first processing component may receive both physiological data from one or more biomarker sensors and voice input from the user, and may use both data streams as inputs to the determination of therapeutic control parameter values before transmitting resulting control instructions to the hardware execution module.

In some embodiments, the general-purpose computing device on which the first processing component is implemented comprises a mobile phone, tablet, laptop computer, or other computing device capable of executing one or more software applications. In some embodiments, a first application executing on the general-purpose computing device is configured to communicate with one or more biomarker sensors through a wireless communication interface and to receive physiological data from those sensors. The first application may additionally provide a user interface through which the user may enter health status information, including physiological parameters, medical history, wellness goals, body measurements, or other health-related information, using any available input mechanism of the computing device, including a touchscreen, keyboard, voice input, or other entry mechanism. In some embodiments, health status information entered by the user through the user interface of the first application, or received from the biomarker sensors, constitutes pre-measured biomarker data that is used as an input to the determination of therapeutic control parameter values. In some embodiments, health status information relevant to the determination of therapeutic control parameter values may additionally reside in a second application executing on the same general-purpose computing device or on a separate device in communication therewith, including without limitation a health maintenance application, a fitness tracking application, a medical records application, or any other application storing physiological or wellness data associated with the user. The first application may be configured to access, retrieve, or receive data from the second application through an application programming interface, a shared data store, an inter-application communication mechanism, or any other data exchange method supported by the operating environment of the computing device, and to incorporate that data into the determination of therapeutic control parameter values. In some embodiments, a third application executing on the general-purpose computing device or on a separate communicatively coupled device is configured to aggregate physiological data received from the first application and health status data received from the second application, to perform therapeutic parameter determination using the aggregated data, and to transmit resulting control instructions or parameter values to the hardware execution module of the controller for conversion into hardware-compatible control outputs. In such embodiments, the first application is responsible for sensor connectivity and data acquisition, the second application is responsible for health status data storage and management, and the third application is responsible for aggregating inputs from both sources and performing or facilitating therapeutic parameter determination for the health-related device. In other embodiments, some or all of these functional responsibilities may be combined within a single application or distributed across any number of applications or devices in communication with one another.

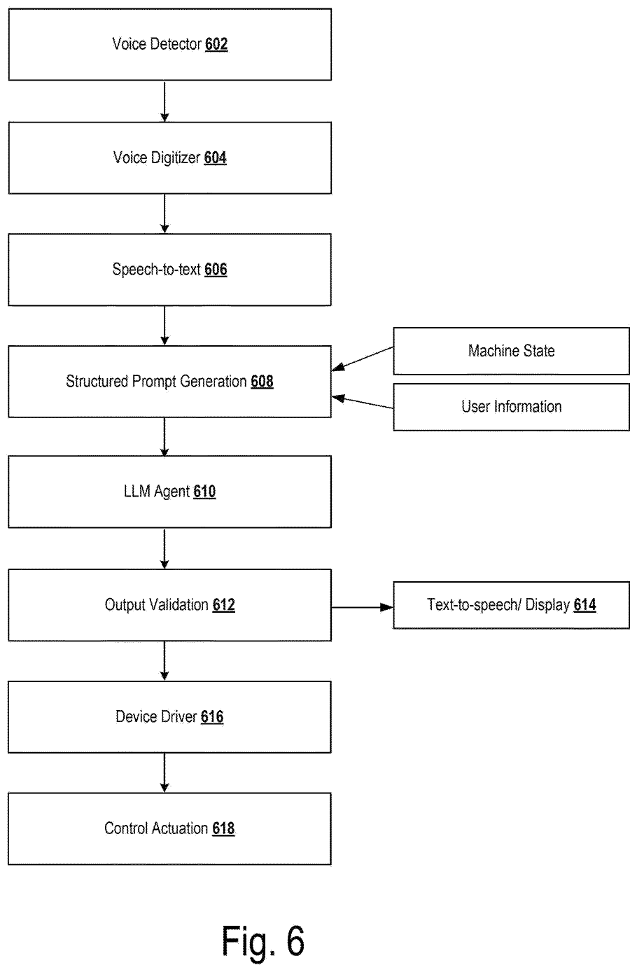

FIG. 6 illustrates an embodiment of a voice-assisted control architecture integrated with the controller 104 for managing operation of the health-related device 106. The embodiment provides structured processing of voice input while maintaining deterministic safety enforcement prior to actuation of device components.

In some embodiments, a voice detector 602 receives acoustic input from a user. The detected voice signal is provided to a voice digitizer 604, which converts the acoustic waveform into structured digital data. The digitized voice data is provided to a speech-to-text module 606, which converts spoken language into textual representations. The textual output may then be processed by a structured prompt generation module 608, which formats the interpreted user input into a structured command representation. In some embodiments, structured prompt generation incorporates contextual data including machine state information, user-specific configuration data, session history, or environmental status information.

The structured prompt is then provided to an LLM agent module 610, which may assist in interpreting user intent, resolving ambiguities, or generating context-aware responses. The LLM agent 610 may incorporate machine state information and user information when generating output. In some embodiments, the LLM agent 610 operates as an assistive interpretive layer and does not directly generate device control signals.

Output generated by the LLM agent 610 is provided to an output validation module 612. The output validation module 612 enforces deterministic safety constraints, predefined thresholds, and device operating limits. In some embodiments, the output validation module 612 compares requested parameter changes against physiological safety thresholds, environmental constraints, and device capability limits. Commands that exceed allowable ranges may be modified, rejected, or replaced with safe default values. Validated control instructions are provided to a device driver module 616, which converts validated commands into hardware-compatible signals suitable for actuation, resulting in control actuation 618.

In some embodiments, a user may state, “Lower temperature to 150 degrees.” The speech-to-text module 606 converts the spoken input into text, and the structured prompt generation module 608 formats the request into a structured representation. The LLM agent 610 confirms that the request corresponds to a thermostat adjustment. The output validation module 612 then compares the requested temperature against predefined safety thresholds and individualized physiological limits; if permissible, the device driver generates control signals that adjust a thermostat setting and modulate voltage or current supplied to a heating element to achieve the setpoint. Deterministic validation remains authoritative to ensure that conversational or LLM-assisted commands do not override safety constraints.

In certain embodiments, the voice input processing and interpretation functions may be performed on a user device such as a phone, tablet, or other computing device, separate from the controller. The separate user device may receive the voice input through its own microphone and convert the voice input into a digital command using speech recognition or other voice interpretation techniques. The digital command may then be transmitted to the controller through a wired or wireless communication connection. In this configuration, the controller remains responsible for controlling operation of the health-related device while the separate device provides the voice interaction interface. The controller may validate the received command and generate corresponding device control outputs.

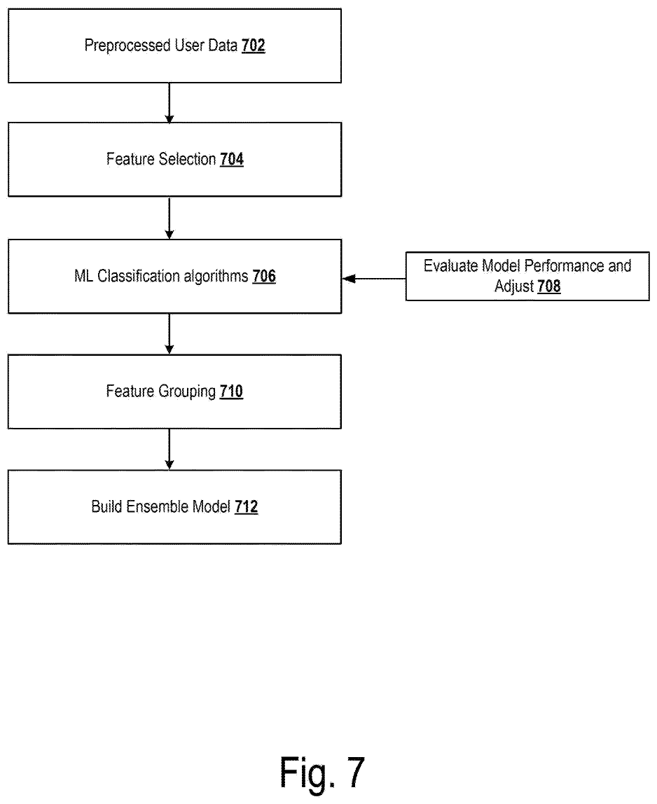

FIG. 7 illustrates an example machine-learning-based physiological modeling architecture used by the controller 104 to determine therapeutic control parameter values for the health-related device 106. The embodiment represents a structured data-processing pipeline configured to transform pre-processed user data into a predictive ensemble model for adaptive control.

In some embodiments, the process begins with pre-processed user data 702, which may include real-time physiological measurements, pre-measured baseline data, environmental measurements, session history, and derived physiological indicators. Pre-processing may include filtering, normalization, windowing, scaling, outlier removal, and timestamp alignment to generate structured input data suitable for computational modeling.

The pre-processed data 702 is provided to a feature selection stage 704, where relevant physiological parameters are identified for modeling. In some embodiments, features may include heart rate, heart rate variability, skin temperature, oxygen saturation, respiration rate, stress indicators, thermal exposure duration, environmental temperature, humidity, or rate-of-change metrics. Feature selection may be performed using statistical ranking methods, correlation analysis, variance analysis, information gain, recursive elimination, or learned importance weighting. In some embodiments, feature selection reduces dimensionality while preserving parameters most predictive of physiological response.

The selected features are then processed by one or more machine-learning classification algorithms 706. These algorithms may include decision trees, support vector machines, logistic regression models, neural networks, Bayesian classifiers, or other non-supervised, supervised or semi-supervised learning techniques. The classification stage may determine physiological state categories such as normal, elevated stress, recovery phase, excessive exposure, cardiovascular strain, or other condition states relevant to therapeutic control.

In some embodiments, the system includes an evaluation module configured to evaluate model performance and adjust parameters 708. The evaluation stage may compute performance metrics such as classification accuracy, confidence scores, error rates, false-positive rates, or cross-validation results. Based on such evaluation, the model parameters, feature weights, or classification thresholds may be updated to improve predictive reliability.

Following classification, the architecture may perform feature grouping 710, wherein correlated or related physiological features are grouped into higher-level composite indicators. For example, cardiovascular stress indicators may combine heart rate, heart rate variability, and blood pressure into a unified stress score. Outputs from multiple classification algorithms 706 and feature groupings 710 are then used to build an ensemble model 712. The ensemble model 712 may combine outputs using weighted voting, averaging, stacking, boosting, or other ensemble techniques. In some embodiments, the ensemble model 712 is also referred to as a physiological and therapeutic model, a user model, or a model.

The resulting ensemble model 712 generates predictive outputs indicative of the user's physiological state and anticipated response to therapeutic stimuli. These predictive outputs are provided to the Therapeutic Parameter Determination Module 218, which determines one or more therapeutic control parameter values for the health-related device 106. In some embodiments, the ensemble model 712 operates in conjunction with deterministic safety constraints such that machine-learning outputs inform but do not override predefined safety thresholds.

The above description of shown example implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. Moreover, the various example implementations described herein may be combined to provide further implementations.

In some embodiments, the method or methods described above may be executed or carried out by a computing system including a tangible computer-readable storage medium, also described herein as a storage machine, that holds machine-readable instructions executable by a logic machine (e.g., a processor or programmable control device) to provide, implement, perform, and/or enact the above described methods, processes and/or tasks. When such methods and processes are implemented, the state of the storage machine may be changed to hold different data.

Since many modifications, variations, and changes in detail can be made to the described preferred embodiments of the disclosure, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A controller for a health-related device, comprising:
at least one processor; and
a memory storing executable instructions that, when executed by the at least one processor, cause the controller to:
receive physiological data from at least one biomarker sensor;
determine at least one therapeutic control parameter value using the physiological data as an input to the determination; and
generate one or more control signals comprising electrical or digital control outputs corresponding to the at least one therapeutic control parameter value, the one or more control signals configured to drive one or more actuation components to modify at least one operational characteristic of a health-related device.

2. The controller of claim 1, wherein the physiological data comprises real-time biomarker data received from the at least one biomarker sensor during use of the health-related device.

3. The controller of claim 1, wherein the physiological data comprises pre-measured biomarker data obtained when the health-related device is not in use by the user.

4. The controller of claim 1, wherein the physiological data comprises a combination of real-time biomarker data received during use of the health-related device and pre-measured biomarker data obtained when the health-related device is not in use by the user.

5. The controller of claim 1, wherein the physiological data includes at least one of body temperature, skin temperature, heart rate, heart rate variability, blood pressure, oxygen saturation, respiratory rate, or metabolic indicators, and wherein the controller is configured to receive the physiological data from at least one of a wearable sensor, an implantable sensor, an ingestible sensor, an optical sensor, or an environmental sensor.

6. The controller of claim 1, wherein the memory further stores a therapeutic parameter range associated with the at least one therapeutic control parameter value, and the controller is further configured to constrain the at least one therapeutic control parameter value within the therapeutic parameter range.

7. The controller of claim 1, wherein the memory further stores physiological characteristics of the user, and wherein the executable instructions further cause the controller to determine the at least one therapeutic control parameter value by performing a calculation using the stored physiological characteristics and the received physiological data as inputs to the determination.

8. (canceled)

9. (canceled)

10. The controller of claim 1, wherein the executable instructions further cause the controller to execute at least one algorithm selected from rule-based logic, statistical analysis, machine learning, or artificial intelligence to determine the at least one therapeutic control parameter value.

11. The controller of claim 1, wherein the controller is configured to control a health-related device comprising at least one of a cold plunge system, a sauna system, a thermal therapy system, an electrical muscle stimulation device, a transcutaneous electrical nerve stimulation device, a massage device, a radiant energy device, a hyperbaric chamber, a rehabilitation device, a fitness recovery device, or a wearable therapeutic device.

12. The controller of claim 1, wherein the one or more control signals are configured to drive actuation components comprising at least one of a pump, a valve, an electromagnetic radiator, a flow guide, an ice generation mechanism, a heating element, a cooling element, a fan, a compressor, a light source, an acoustic emitter, a vibration source, a pressure-modulating device, or a relay configured to selectively connect an electrical source to a load within the health-related device.

13. The controller of claim 1, wherein the controller is distributed across a first processing component and a second processing component, wherein the first processing component is implemented on a computing device communicatively coupled to the at least one biomarker sensor through a communication interface, wherein the first processing component receives the physiological data from the at least one biomarker sensor and transmits the physiological data or a processed representation thereof to the second processing component through a communication connection, and wherein the second processing component is implemented on a hardware execution module operatively coupled to the health-related device.

14. (canceled)

15. (canceled)

16. The controller of claim 13, wherein the first processing component is configured to execute one or more software applications, the one or more software applications collectively configured to: receive physiological data from the at least one biomarker sensor through a communication interface; receive health status information associated with the user, the health status information comprising at least one of user-entered data received through a user interface of the computing device, data retrieved from one or more other applications executing on the computing device or on a device in communication therewith, or pre-measured or real-time biomarker data; and use the physiological data and the health status information as inputs to the determination of the at least one therapeutic control parameter value, and transmit resulting control instructions to the second processing component.

17. A controller for a health-related device, comprising:
at least one processor; and
a memory storing executable instructions that, when executed by the at least one processor, cause the controller to:
receive voice input;
determine at least one therapeutic control parameter value using the voice input as an input to the determination; and
generate one or more control signals comprising electrical or digital control outputs corresponding to the at least one therapeutic control parameter value, the one or more control signals configured to drive one or more actuation components to modify at least one operational characteristic of a health-related device.

18. The controller of claim 17, wherein the memory further stores a therapeutic parameter range associated with the at least one therapeutic control parameter value, and the controller is further configured to constrain the at least one therapeutic control parameter value within the therapeutic parameter range.

19. The controller of claim 17, wherein the memory further stores physiological characteristics of the user, and wherein the executable instructions further cause the controller to determine the at least one therapeutic control parameter value by performing a calculation using the stored physiological characteristics and the received voice input as inputs to the determination.

20. (canceled)

21. (canceled)

22. The controller of claim 17, wherein the executable instructions further cause the controller to convert the voice input into a textual representation using a speech recognition process prior to determining the at least one therapeutic control parameter value.

23. The controller of claim 22, wherein the executable instructions further cause the controller to interpret the textual representation using a large language model to determine user intent or a requested therapeutic adjustment, and to use an output of the large language model as an input to the determination of the at least one therapeutic control parameter value.

24. (canceled)

25. The controller of claim 17, wherein the voice input is received by a separate device communicatively coupled to the controller through a communication connection, and wherein the separate device converts the voice input into a digital command and transmits the digital command to the controller, and wherein the controller validates the digital command and generates the one or more control signals in response thereto.

26. The controller of claim 25, wherein the controller is distributed across a first processing component implemented on the separate device and a second processing component implemented on a hardware execution module operatively coupled to the health-related device, wherein the first processing component receives the voice input and transmits control instructions to the second processing component through a communication connection, and wherein the hardware execution module generates electrical or digital outputs for driving the one or more actuation components in response to the received control instructions.

27. (canceled)

28. The controller of claim 17, wherein the executable instructions further cause the controller to receive physiological data from at least one biomarker sensor in addition to the voice input, and to use at least one of the voice input and the physiological data as an input to the determination of the at least one therapeutic control parameter value.

29. A method for controlling operation of a health-related device, comprising:
receiving physiological data from at least one biomarker sensor;
determining, by a controller comprising at least one processor, at least one therapeutic control parameter value using the physiological data as an input to the determination; and
generating, by the controller, one or more control signals comprising electrical or digital control outputs corresponding to the at least one therapeutic control parameter value, the one or more control signals configured to drive one or more actuation components to modify at least one operational characteristic of the health-related device.

30. The method of claim 29, wherein the physiological data comprises real-time biomarker data received from the at least one biomarker sensor during use of the health-related device.

31. The method of claim 29, wherein the physiological data comprises pre-measured biomarker data obtained when the health-related device is not in use by the user.

32. The method of claim 29, wherein the physiological data comprises a combination of real-time biomarker data received during use of the health-related device and pre-measured biomarker data obtained when the health-related device is not in use by the user.

33. (canceled)

34. The method of claim 29, wherein the controller is distributed across a first processing component and a second processing component, and further comprising receiving the physiological data at the first processing component through a communication interface from the at least one biomarker sensor, performing at least one processing operation on the physiological data at the first processing component, and transmitting the physiological data or a processed representation thereof from the first processing component to the second processing component.

35. A method for controlling operation of a health-related device, comprising:
receiving voice input;
determining, by a controller comprising at least one processor, at least one therapeutic control parameter value using the voice input as an input to the determination; and
generating, by the controller, one or more control signals comprising electrical or digital control outputs corresponding to the at least one therapeutic control parameter value, the one or more control signals configured to drive one or more actuation components to modify at least one operational characteristic of the health-related device.

36. The method of claim 35, further comprising converting the voice input into a textual representation using a speech recognition process prior to determining the at least one therapeutic control parameter value.

37. The method of claim 36, further comprising interpreting the textual representation using a large language model to determine user intent or a requested therapeutic adjustment, and using an output of the large language model as an input to the determination of the at least one therapeutic control parameter value.

38. (canceled)

39. The controller of claim 13, wherein the first processing component is implemented on a mobile phone that receives the physiological data from the at least one biomarker sensor through a wireless communication interface, accesses health status information associated with the user, and uses the physiological data and the health status information as inputs to the determination of the at least one therapeutic control parameter value, and transmits control instructions to the second processing component through a communication connection; and wherein the second processing component receives environmental data from at least one environmental sensor associated with the health-related device and generates electrical or digital outputs for driving at least one actuation component using the control instructions and the environmental data.

40. (canceled)

42. The method of claim 35, wherein the first processing component is implemented on a mobile phone that receives voice input, accesses health status information associated with the user, and uses the voice input and the health status information as inputs to the determination of the at least one therapeutic control parameter value, and transmits control instructions to the second processing component; and wherein the second processing component receives environmental data from at least one environmental sensor associated with the health-related device and generates electrical or digital outputs for driving at least one actuation component using the control instructions and the environmental data.
